(19) United States
(12) Patent Application Publication
(10) Pub. No.: US 2017/0238964 A1
(43) Pub. Date: Aug. 24, 2017

(54) SET OF INTRODUCERS FOR USE IN PLACEMENT OF SUBURETHRAL SLING

(71) Applicant: GEORGE MURRAY BELL, JOHANNESBURG (ZA)

(72) Inventor: GEORGE MURRAY BELL, JOHANNESBURG (ZA)

(21) Appl. No.: 15/214,306

(22) Filed: Jul. 19, 2016

(30) Foreign Application Priority Data
Feb. 19, 2016 (ZA) F201600224

(51) Int. Cl. A61B 17/34; A61F 2/00

(52) U.S. Cl. CPC A61B 17/3468; A61F 2/0045; A61B 2017/00805

(57) ABSTRACT

The present invention relates to the treatment of persons with urinary incontinence. More particularly, the invention extends to a set of introducers (also commonly referred to as tunnelers) shaped to accommodate the Altis® suburethral sling (Coloplast Corporation), which is commonly used in surgical treatment of female stress urinary incontinence.

### FIELD OF THE INVENTION

This invention relates to treatment of persons with urinary incontinence. More particularly, the invention extends to an introducer (also commonly referred to as a tunneler) shaped to accommodate the Altis® suburethral sling (Coloplast Corporation), which is commonly used in surgical treatment of female stress urinary incontinence.

### BACKGROUND OF THE INVENTION

Involuntary loss of urine is often referred to as urinary incontinence. This condition is more frequently experienced by women than by men.

Weak or overactive bladder muscles and weakness of sphincter muscles supporting the urethra may contribute to urinary incontinence in women. It has also been reported that pregnancy, childbirth, menopause, and nerve damage can be contributors, leading to female stress urinary incontinence.

Laughing, sneezing, coughing, lifting, exercising, and even sexual intercourse may lead to urine involuntarily escaping from a patient diagnosed with urinary stress incontinence.

Fortunately, single-incision mini-sling systems were developed to treat female stress urinary incontinence during minimally invasive surgical procedures.

Most single-incision mini-sling systems consist of a small suburethral sling, manufactured from a synthetic material such as polypropylene surgical mesh; and two introducers (tunnelers), which are hand-held surgical placement instruments adapted to facilitate correct placement of the sling during the surgical procedure. The introducers are shaped and configured to be mirror images of each other.

The sling placement procedure corrects stress incontinence by placing, with the aid of the introducers, the small suburethral sling beneath the urethra. Like a cradle, the sling forms a substructure, which supports the urethra from behind during straining maneuvers or other activities to prevent leakage from occurring.

While known sling placement procedures, in which the ubiquitous Altis® introducers are used with the Altis® suburethral sling, have shown some success they do, however, have restrictions, especially where the patient is a larger woman. Cases have been reported where urologists struggle to position the sling behind the urethra when they perform this surgical procedure on obese woman using the ubiquitous Altis® introducers. The reason for this lies in the fact that the existing Altis® introducers are not shaped to cater for bodies of larger woman.

Accordingly, introducers for more effective placement of suburethral slings in treatment of female stress urinary incontinence associated with larger woman, remain a need.

The applicant believes that the set of introducers as described in this specification is capable of addressing, at least in part, the above mentioned problems associated with current Altis® introducers.

### SUMMARY OF THE INVENTION

In accordance with this invention there is provided a set of surgical introducers for introducing a suburethral sling into the human body.

Each introducer is a mirror image of the other and comprises a tine having proximal and distal ends, the tine is connected to a curved wire-like body having: a first portion extending from a handle end to an elbow; and a second portion extending from the elbow to the tine.

The tine is narrower than the wire-like body and includes a sharp tip at the distal end. The first portion extends along an axis parallel to a longitudinal axis of the handle while the second portion is bent relative to the first portion at the elbow to define a semi-circular concave shape terminating at the proximal end of the tine. The set of surgical introducers are characterized in that an imaginary rectilinear line, slanting from the elbow towards the distal end, can be visualized.

Preferably, there is provided for the distance B between the elbow of each introducer and the distal end of each introducer to be between about 60 mm and 75 mm.

The invention also extends to use of the set of introducers in a method of treatment of a female patient diagnosed with stress urinary incontinence.

### BRIEF DESCRIPTION OF THE DRAWINGS

An embodiment of the invention is now described, by way of example, with reference to the accompanying drawings. In the drawings:

FIG. 1 is a front perspective view of a set of sharp ended tine surgical tunnelers or introducers, which comprise left and right tunnelers wherein each one of the tunnelers has a substantially semi-circular configuration subtending a 180° arcuate extent;

FIG. 2 is a rear perspective view thereof;

FIG. 3 is a first side elevational view thereof;

FIG. 4 is a second side elevational view thereof;

FIG. 5 is a first end view thereof as seen from the tine end of the tunneler;

FIG. 6 is a second end view thereof as seen from the handle end of the tunneler;

FIG. 7 is a top plan view thereof; and

FIG. 8 is a bottom plan view thereof.

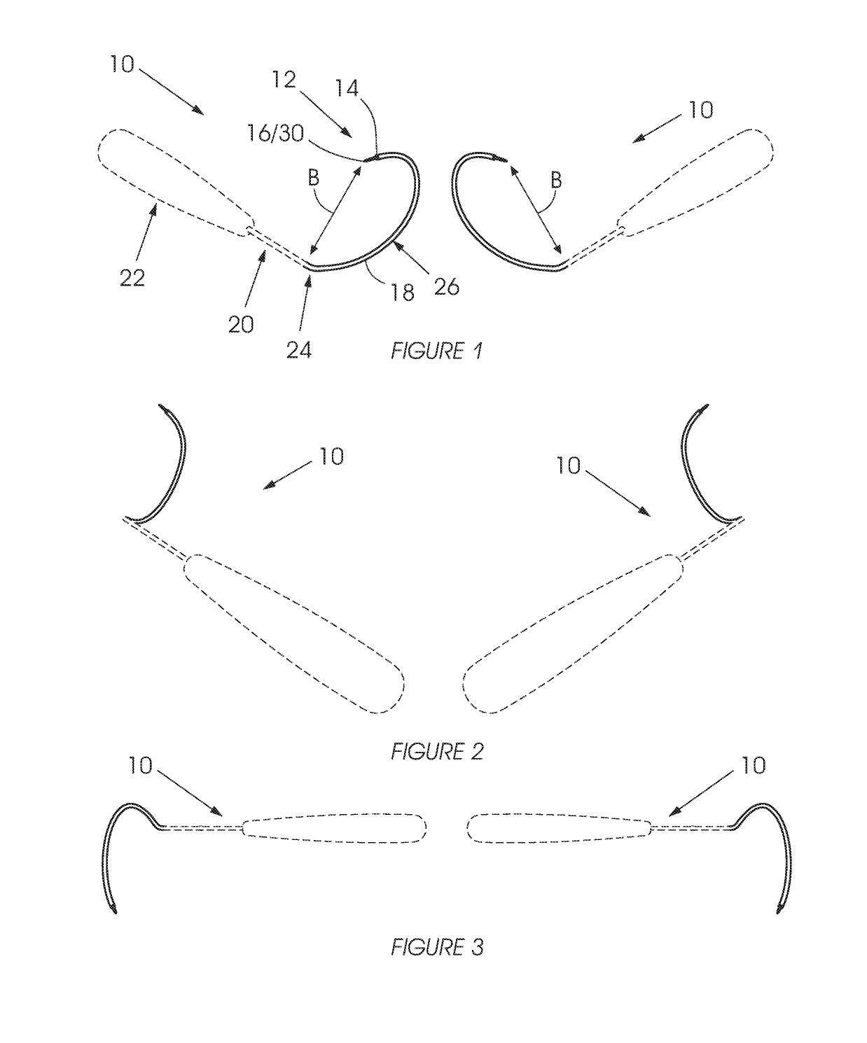

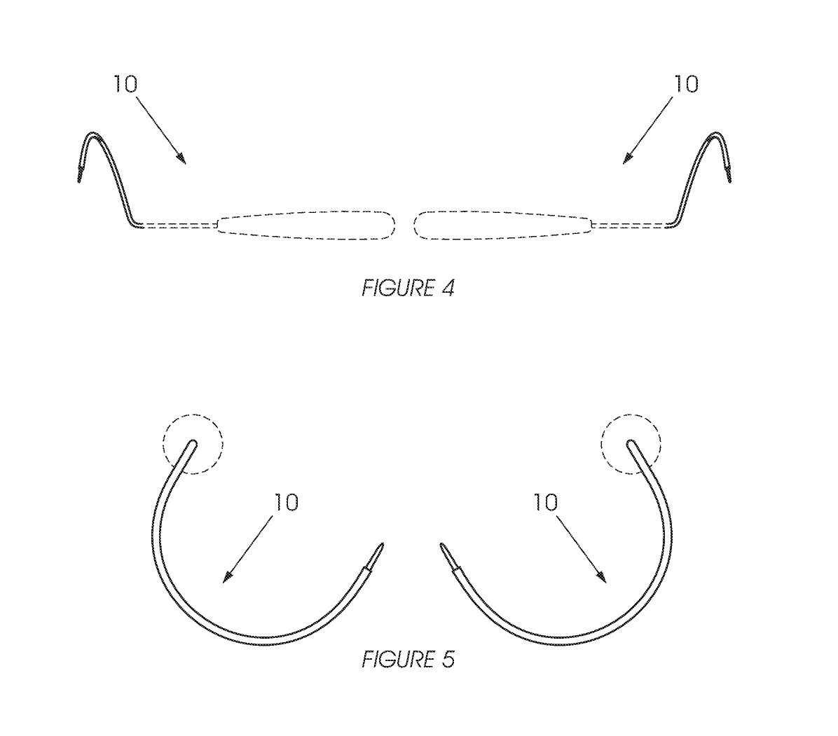

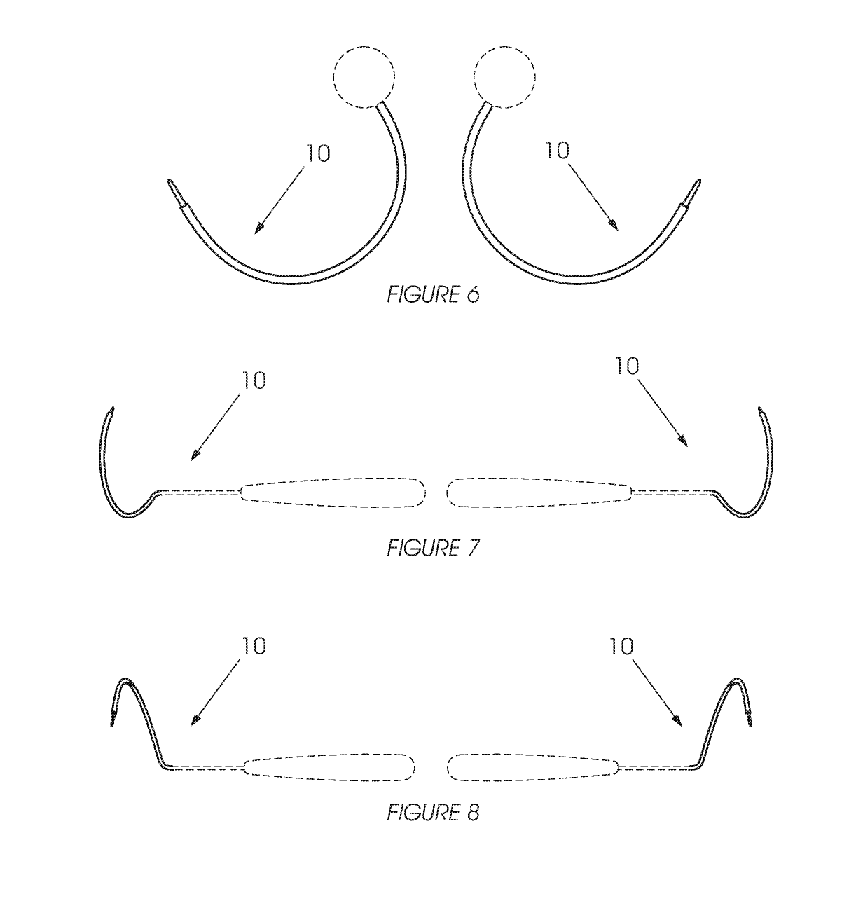

### DETAILED DESCRIPTION OF THE INVENTION

This description is presented in the cause of providing what is believed to be the most useful and readily understood description of the principles and conceptual aspects of the invention. In this regard, no attempt is made to show operational details of the invention in more detail than is necessary for a fundamental understanding of the invention, the description taken with the Figures making apparent to those skilled in the art how at least one form of the invention may be embodied in practice.

A variety of so-called tunnelers or surgical introducers are known and used to introduce medical devices into the human body. However, in the treatment of female patients diagnosed with stress urinary incontinence, the Altis® introducer is commonly employed to deliver and correctly place an anchor end of a single incision Altis® sling to the female human body. The Altis® sling is made from a polypropylene light-weight mesh flanked by two anchor ends that anchors the sling to tissue of the human body after transobturator placement thereof during a surgical procedure.

Although, the Altis® introducer is often successfully employed to introduce the Altis® sling to the female human body, it fails to do so where larger woman is treated for stress urinary incontinence. The reason behind this is because the pubic ramus and overlying fat and tissue in larger woman are often disposed such that correct placement of the anchor ends of the Altis® sling cannot be effected without difficulty when the standard Altis® introducer is employed because of the specific curvature of the Altis® introducer.

Referring to the drawings, a set of surgical tunnelers or introducers (10) in accordance with the invention and for introducing a suburethral sling into the female human body, is provided. Each introducer (10) is a mirror image of the other and adapted to be used on the left and right side of the patient during surgery.

Each introducer comprises a tine (12) having proximal (14) and distal (16) ends, the tine (12) is connected to a curved wire-like body (18) which is divided into first (20) and second (26) portions. The first portion (20) extends from a handle (22) end to an elbow (24), while the second portion (26) extends from the elbow (24) to the tine (12).

The tine (12) is narrower than the wire-like body (18) and includes a sharp tip (30) at the distal end (16). Both the tine (12) and the wire-like body (18) are typically manufactured from a metal alloy while the handle is manufactured from a suitable plastic.

The first portion (20) extends along an axis parallel to a longitudinal axis of the handle (22) while the second portion (26) is bent relative to the first portion (20) at the elbow (24) to define a semi-circular concave shape, terminating at the proximal end (14) of the tine (12). The set of surgical introducers (10) are characterized in that an imaginary rectilinear line B, slanting from the elbow (24) towards the distal end (16), can be visualized.

In use, the sharp tip (30) at the distal end (16) of the tine (12) is pushed to project partially through the anchor end (not shown) of the sling so that the anchor end is accommodated on the distal end (16) with the sharp tip (30) extending partially through the anchor end. At this stage, the introducer (10) with the anchor end positioned thereon is maneuvered through a 1 to 2 cm vaginal incision, while the index finger protects the bladder of the patient.

The hand-held introducer is then further maneuvered until the distal end (16) is brought up to the medial border of the descending pubic ramus. The introducers (10) of the invention, each of which defines a distance B, of anywhere between about 60 and 75 mm, between the elbow (24) and the sharp tip (30), easily reach into the obturator foramen and past the descending pubic ramus thereby greatly facilitating swift and correct placement of the anchor ends within the body.

Thereafter, a one quarter turn rotation of the handle (22) allows the anchor end at the sharp tip (30) to easily protrude through the obturator membrane and/or muscles. The result is that the sharp tip (30) then faces towards the surgeon from within the body of the patient, and at this point the surgeon knows that the anchor end penetrated through the entire obturator membrane such that the sling is in the preferred suburethral position within the female body.

Subsequently, the sling is tensioned, incision closed and the combination of sling and tissue ingrowth under the urethra becomes the new substructure for urethral support.

While one embodiment of the present invention has been herein shown and described, it will be understood that minor changes may be made without departing from the spirit and scope of the invention.

The monopoly for which protection is claimed is set out in the set of appended claims which follows hereinafter.

1. A set of surgical introducers for introducing a suburethral sling into the female human body, each introducer being a mirror image of the other and comprising a tine having proximal and distal ends, the tine being connected to a curved wire-like body having:
a first portion extending from a handle end to an elbow; and
a second portion extending from the elbow to the tine,
the tine being narrower than the wire-like body and includes a sharp tip at the distal end, the first portion extending along an axis parallel to a longitudinal axis of the handle with the second portion being bent at the elbow relative to the first portion to define a semi-circular concave shape terminating at the proximal end of the tine, wherein the set of surgical introducers are characterized in that an imaginary rectilinear line, slanting from the elbow towards the distal end, can be visualized.

2. The set of surgical introducers as claimed in claim 1, wherein the distance between the elbow of each introducer and the distal end of each introducer is about 60 mm.

3. The set of surgical introducers as claimed in claim 1, wherein the distance between the elbow of each introducer and the distal end of each introducer is between about 60 mm and 75 mm.

4. A set of surgical introducers as claimed in claim 1, for use in a method of treatment of a female patient diagnosed with stress urinary incontinence, which treatment comprises making a vaginal incision, accommodating anchor ends of a mini-sling on sharp tips of the respective introducers, guiding the tips through the incision and placing the mini-sling suburethrally in a supportive position relative to the urethra.
